(19) United States
(12) Patent Application Publication
(10) Pub. No.: US 2026/0198908 A1
(43) Pub. Date: Jul. 16, 2026

(54) METHOD AND DEVICE FOR TRANSPERINEAL PROSTATE BIOPSY

(71) Applicant: MULTI-SCALE MEDICAL ROBOTICS CENTER LIMITED, Hong Kong (CN)

(72) Inventors: Zheng LI, Hong Kong (CN); Xiao LUO, Hong Kong (CN); Ka Fung Peter CHIU, Hong Kong (CN); Man Cheong LEI, Hong Kong (CN); Yunhui LIU, Hong Kong (CN); Wai Yan Philip CHIU, Hong Kong (CN); Chi Fai NG, Hong Kong (CN)

(21) Appl. No.: 19/136,421

(22) PCT Filed: Dec. 8, 2023
(86) PCT No.: PCT/IB2023/062390

§ 371 Date: Jun. 6, 2025

Related U.S. Application Data
(60) Provisional application No. 63/386,673, filed on Dec. 9, 2022.

(51) Int. Cl. A61B 10/02; A61B 8/00; A61B 8/12; A61B 17/34; A61B 34/30

(52) U.S. Cl. CPC A61B 10/0241; A61B 8/12; A61B 8/4218; A61B 8/54; A61B 17/3403; A61B 34/30; A61B 2017/3409; A61B 2017/3413; A61B 2034/303

(57) ABSTRACT

This invention relates to method and device for transperineal prostate biopsy. In one embodiment, said device for transperineal prostate biopsy comprises: (a) a first parallel mechanism for generating translational or rotational motion of an ultrasound probe; (b) a series-parallel hybrid mechanism as an end-effector of said first parallel mechanism for housing said ultrasound probe and a biopsy needle-guide; (c) a drive module of said series-parallel hybrid mechanism for generating rotational motion and translational motion of said ultrasound probe along said ultrasound probe's axis; (d) a second parallel mechanism of said series-parallel hybrid mechanism for controlling the configuration of said biopsy needle-guide relative to said ultrasound probe and confining trajectory of said biopsy needle to an axial imaging plane of said ultrasound probe; and a controller for executing a kinematic modelling method.

### FIELD OF THE INVENTION

The present invention generally relates to transperineal prostate biopsy and particularly to a novel mechanism that can be used to assist surgeons to perform a transperineal prostate biopsy, positioning of biopsy needle based on this device and surgical workflow of transperineal prostate biopsy with the invented device.

### BACKGROUND OF THE INVENTION

Prostate cancer is the second most common cancer after breast cancer and is one of the main causes of male death. Early and reliable detection of prostate cancer has a huge impact on the successful treatment of high-risk patients and on avoiding overtreatment in low-risk patients. Commonly, the most reliable technique to detect prostate cancer and estimate its aggressiveness is prostate biopsy.

There are three types of prostate biopsy in clinical practice. An important distinction is where the needle passes through. Since the puncture sites include urethra, perineum and rectum, the prostate biopsy is generally divided into transrectal, transperineal, and transurethral biopsies. The transrectal ultrasound guided prostate biopsy, as the common way of diagnosing prostate cancer, requires an ultrasound probe inserted into the rectum to show a suspicious area. Then the biopsy needle punctures through the anterior rectum wall to the prostate. However, the transrectal prostate biopsy is prone to several complications such as rectal bleeding and infections. Moreover, biopsy needles are difficult to cover the whole prostate. This method has been previously described in several systems, such as in U.S. Pat. No. 9,877,788B2, entitled “MRI-safe robot for transrectal prostate biopsy” filed on Jan. 30, 2018, and in U.S. Pat. No. 10,368,850B2, entitled “System and method for real-time ultrasound guided prostate needle biopsies using a compliant robotic arm” filed on Aug. 6, 2019. As for the transurethral prostate biopsy, in order to ensure safety, the needle is usually displayed in the ultrasonic imaging plane in real-time. Therefore, this biopsy method is difficult to use ultrasonic real-time imaging, and it is difficult to ensure its safety. In addition, there are too many puncture positions passing through the urethral wall, so there is a great risk of complications. Transperineal prostate biopsy inserts the biopsy needle through the skin at the perineum under the guidance of an ultrasound probe. And transperineal biopsy could be safer, ensure lower infection risks. Instead of undergoing general anesthesia, in this method, patients only need to be anesthetized locally. Therefore, in this situation, the site for biopsy does not need a harsh medical environment, which can improve the overall biopsy efficiency.

A typical transperineal prostate biopsy is guided by transrectal ultrasound-guided images. The surgeon inserts the biopsy needle into the prostate through the guide template with the guidance of the acquired ultrasound images. The guide template is equipped with a series of through-holes parallel to the probe. A transperineal biopsy needle will be inserted through these holes to enter the interest target. The surgeon needs to fix the probe with one hand and insert the biopsy needle with the other hand. The result of this procedure is highly dependent on the experience of the surgeon, which makes the process of prostate biopsy difficult to define and follow. In order to improve the accuracy and treatment quality of puncture intervention. It is significant to develop a flexible robotic device system that undertakes the positioning and aiming work during the process of prostate biopsy, and in this way, surgeons only need to perform the manual needle insertion with the assistance of the designed device.

It is worth mentioning that the clinical robotic prostate biopsy workflow includes preoperative work and intraoperative work. Since prostate cancer is a heterogeneous multi-focal disease, it is important to confirm the cancer's suspicious regions with the help of cancer-image-guided targeting. Magnetic resonance imaging (MRI) can provide the highest contrast and spatial resolution for prostate anatomy. Therefore, during the process of preoperative work, an MRI scan is performed on the patients, and MRI images of the prostate are segmented and reconstructed in 3 dimensions. However, the real-time performance of MRI images is relatively poor. In addition, MRI is very demanding in the working environment and has high antimagnetic requirements for other equipment. It is inconvenient for surgeons to conduct a biopsy in the working environment of MRI. The transrectal ultrasound (TRUS) scan can provide real-time images and it is convenient to use. One of the simplest methods of biopsy targeting is MRI-TRUS image fusion. Therefore, the ultrasonic probe is moved to the rectum and scanned to obtain a series of 2D ultrasonic images, and the prostate region is segmented and reconstructed in 3D. Next Registration and fusion of MRI and ultrasonic 3D models are conducted. What's more, in terms of the intraoperative process, the ultrasonic probe is held to the appropriate position while the physician selects the target lesion point. The assisted device performs planning and provides alignment of a needle guide for biopsy. And then surgeons insert the needle and perform the biopsy.

Based on this method mentioned above, some commercial devices (such as Monalisa, Artemis) have designed their robotic system for prostate biopsy. For example, Monalisa adopts transperineal biopsy with the guidance of MRI-TRUS guided images. However, it lacks real-time in-line ultrasound monitoring of needle tracks as the probe cannot be in-line with the biopsy needle. Therefore, the exacting needle position cannot be seen on the ultrasound image window, leading to reduced quality control and safety of the system. The probe has only two degrees of freedom and is not flexible enough. It is necessary for the probe to yaw or pitch during the process of biopsy in order to avoid the obstruction of the pubic. In terms of Artemis, its puncture points of Artemis are different between each other. Thus, there have no RCM (remote center of motion) constraint. It is worth mentioning that using a small number of puncture positions can reduce patient trauma. Based on the robot arm, the trans-perineal biopsy needle guide has certain limitations in reaching various regions of the prostate, e.g., extreme anterior due to pubic bone obstruction. It seems more operator-dependent in targeting and the device of the arm only seems to provide the primary position beside the rotation degree of freedoms.

Since the volume of prostate is about 75 ml, the corresponding workspace of the robotic device should cover the volume of prostate. Considering the workspace and the working environment, the device′ volume should be relatively compact. The system should provide enough support force when the surgeon inserts the biopsy needle. The biopsy needle should be observed in the real-time image plane of the probe to ensure the safety. All the factors contribute a complete transperineal biopsy system.

Hitherto, very few flexible interventional devices for transperineal prostate biopsy have been designed. It would be a significant advantage to introduce robotic devices into the such system, which can perform the aiming work accurately with RCM constraints. The device can have a compact volume, sufficient stiffness and degrees of freedom. Accordingly, there is a need in the art for flexible assistance biopsy devices to improve the efficiency and accuracy of prostate biopsy.

### SUMMARY OF THE INVENTION

This invention accomplishes the aforementioned objective by providing a transperineal prostate biopsy device with functions controlling probe configuration and controlling needle configuration, and the surgical workflow with MRI-TRUS guided images to target the cancer suspicious regions. The flexible device comprises a lower half parallel mechanism and an upper half series-parallel hybrid mechanism: the upper part of the device includes a drive module for generating rotational motion and translational motion of the probe along with its axis, and a parallel mechanism for controlling the configuration of the needle relative to the probe. The lower part of the device includes two designed universal joints with four driver actuators for generating rotational motion and translation motion of the upper part.

In one aspect of the invention, the device can guarantee two RCM constraints including RCM-probe and RCM-needle synchronously. The device has a total of 8 degrees of freedom. The probe fixed on the device will have 6 degrees of freedom based on the integration of the upper and the lower part of the device, including three rotational movements and translational motions in three directions. RCM-probe is located at the patient's anus, and the probe has no relative movement with respect to the patient's anus. The configuration of the needle guide related to the probe is determined by a two degree of freedom parallel mechanism. The RCM-needle position could be movable relative to the probe under different working conditions.

More particularly, based on the characteristic of the invented device for transperineal prostate biopsy, the kinematic analysis is established to deal with RCM-probe and RCM-needle constraints and to describe the relationship between the displacements of all the actuators or the motors and the target position.

In another aspect of the invention, the device could hold the ultrasonic probe to the appropriate configuration with RCM-probe constraint and then achieve a certain trajectory to acquire the required ultrasound images. Based on the previous MRI images, the target points will be obtained by the result of 3D fusion and registration of the MRI images and the acquired ultrasound images.

With the invented device, the surgical workflow can be described as follows. First, the ultrasonic probe is held by the device to conduct the specific configuration with RCM-probe constraint and then a series of ultrasound images are acquired. Next, based on the fusion and registration of the MRI and ultrasound images, the cancer suspicious regions are targeted. As a consequence, the device performs the aiming work with two RCM constraints, at the same time, the plane of movement of the needle will always be limited to the imaging plane of the probe. Finally, the surgeon performs the manual needle insertion through the perineal to the prostate of the patient.

The invention and its features may be best understood in detail with the following description and illustrative drawings.

### BRIEF DESCRIPTION OF THE DRAWINGS

FIG. 1 illustrates an orthogonal view of the prostate biopsy device according to the features of the propose invention.

FIG. 2A illustrates the upper part of the prostate biopsy device which can perform the rotation and translation of the probe relative to its axis and the movement of the biopsy needle in the image plane relative to the ultrasound probe according to the features of the present invention.

FIG. 2B is a partial sectional view of the upper part of the prostate biopsy device according to the features of the present invention.

FIG. 3A illustrates an orthogonal view of the passive motion module of the lower part of the prostate biopsy device. These mainly include two designed universal joints.

FIG. 3B is a partial sectional view of the passive motion module of the lower part of the prostate biopsy device according to the features of the present invention.

FIG. 4 illustrates a kinematic diagram of the prostate biopsy device with partial view according to the features of the propose invention.

FIG. 5 illustrates the RCM constraints during the biopsy process of the device, which include the needle-RCM constraint and the probe-RCM constraint according to the features of the present invention.

FIG. 6 illustrates a kinematic diagram of the lower part of the device according to the features of the present invention.

FIG. 7A are kinematic diagram of the whole device according to the features of the present invention.

FIG. 7B are motion diagram of the whole device under two RCM constraints according to the features of the present invention.

FIG. 8 illustrates an orthogonal view of the upper part of the prostate biopsy device when the probe performs rotation relative to its axis and translation according to the features of the present invention.

FIGS. 9A and 9B are the orthogonal views of the needle motion module of the upper part of the prostate biopsy device when the needle performs the tilt motion relative to the probe of the device according to the features of the present invention.

FIGS. 10A and 10B are the orthogonal views of the needle motion module of the upper part of the prostate biopsy device when the needle performs the translation motion relative to the probe of the device according to the features of the present invention.

FIGS. 11A and 11B are the orthogonal views of the prostate biopsy device when it performs the yaw motion according to the features of the present invention.

FIGS. 12A and 12B are the orthogonal views of the prostate biopsy device when it performs the pitch motion according to the features of the present invention.

FIG. 13 shows the workflow with the invented device for transperineal prostate biopsy according to the features of the present invention.

FIGS. 14A to 14C illustrate another mechanism of the needle motion module that can make the needle perform the tilt motion and translation in the image plane relative to the probe of the device according to the features of the present invention.

FIG. 15 illustrates the advantages of invented device compared to traditional methods during transperineal prostate biopsy procedure.

### DETAILED DESCRIPTION OF THE INVENTION

A novel transperineal prostate biopsy device is designed to improve efficiency, accuracy and treatment quality. This device is able to undertake the positioning and aiming work during the process of prostate biopsy under the guidance of the MRI-Ultrasound fusion images, and in this way, surgeons only need to perform the manual needle insertion, which makes the process less operator dependent. Based on this robotic system, the biopsy needle will be inserted through the patient's perineal and always be observed based on the ultrasound real-time images, which could be safer and ensure lower infection risks. Instead of undergoing general anesthesia, patients only need to be anesthetized locally. The site for the biopsy does not need a harsh medical environment, which can improve the overall biopsy efficiency. Besides, the device could hold the ultrasonic probe to the appropriate position and then achieve a certain trajectory to acquire the required ultrasound images. Based on the previous MRI images, the target points will be obtained by the result of 3D fusion and registration of the MRI images and the acquired ultrasound images. After given the target point, the device will perform its motion to realize the aiming work.

This device has a total of 8 degrees of freedom. The probe fixed on the device will have 6 degrees of freedom, including three rotational movements and translational motions in three directions. The position of the needle guide related to the probe could be determined by a two degree of freedom parallel mechanism. Furthermore, based on the flexible structure, the whole system includes two RCM (remote center of motion) constraints, namely RCM-needle and RCM-probe. The RCM-needle position could be movable relative to the probe under different working conditions.

Main advantages of this device include: It is more rigid than the serial robot, and adopts a serial-parallel hybrid method; The imaging plane of the probe is always on the same plane as the needle to ensure safety; The device is flexible and guarantees 8 degrees of freedom in a compact case, and can cope with the constraint workspace; The whole system can ingeniously guarantee two RCM constraints; The probe of the device have multiple degrees of freedom and RCM-probe constraints are also guaranteed; The RCM-probe positions and RCM-needle positions can be movable relative to patients.

This invention provides a transperineal prostate biopsy device. In one embodiment, said transperineal prostate biopsy device comprises: a) a parallel mechanism with two designed universal joints for generating ultrasound probe's translation including horizontal and vertical movement and rotational motion including pitch and yaw motion with RCM-probe constraint; b) a series-parallel hybrid mechanism as the end-effector of said parallel mechanism for housing said ultrasound probe and biopsy needle-guide; c) a drive module of said series-parallel hybrid mechanism for generating rotational motion and translational motion of said ultrasound probe along its axis; a parallel mechanism of said series-parallel hybrid mechanism for controlling the configuration of needle-guide relative to said ultrasound probe and confining the trajectory of the biopsy needle to the axial imaging plane of said probe; d) a kinematic modeling method for guaranteeing said RCM-probe constraint based on said parallel mechanism and said drive module and guaranteeing said RCM-probe constraint and RCM-needle constraint simultaneously based on said parallel mechanism and said series-parallel hybrid mechanism.

In one embodiment, said parallel mechanism including two said designed universal joints and two designed linear motion modules with 4 DoF; said series-parallel hybrid mechanism connected to said 4-DoF parallel mechanism, further including said ultrasound probe, said drive module, said parallel mechanism with 2 DoF connected to said drive module; said 2-DoF parallel mechanism including two lifting modules and said needle-guide. Wherein said series-parallel hybrid mechanism as the upper part of the invented device is used as the end-effector of said 4-DoF parallel mechanism which is regarded as the lower part of the invented device.

In one embodiment, said linear motion modules of said 4-DoF parallel mechanism, further including a 2-DoF front motion module and a 2-DoF rear motion module.

In one embodiment, said 2-DoF front motion module, further including a horizontal motion module and a vertical motion module, said horizontal motion module connected said vertical motion module, said a universal joint connected said horizontal motion module.

In one embodiment, said 2-DoF rear motion module, further including a horizontal motion module and a vertical motion module, said horizontal motion module connected said vertical motion module, said another universal joint connected said horizontal motion module.

In one embodiment, said drive module, further including a chamber to fix said ultrasound probe.

In one embodiment, said 2-DoF parallel mechanism controls the configuration of said needle-guide relative to said probe, said needle-guide can perform translation and tilt motion relative to said probe.

In one embodiment, said 2-DoF parallel mechanism connected said chamber.

In one embodiment, said 2-DoF parallel mechanism, further including another mechanism to make said needle perform the tilt motion and translation in the image plane relative to said probe, which comprises: a parallelogram mechanism connected to said chamber; a needle guide for guiding the biopsy needle connected to said parallelogram; two motors connected to the two joints of parallelogram mechanism.

In one embodiment, said 2-DoF parallel mechanism with said needle-guide, make the moving plane of said needle coincide with the imaging plane of said probe.

In one embodiment, said universal joints of said 4-DoF parallel mechanism, further including a front and a rear universal joint.

In one embodiment, said front universal joint, further including a joint support, a pitch rotating part, a yaw rotating part. Said joint support connected said horizontal motion module of said front motion module.

In one embodiment, said rear universal joint, further including a joint support, a pitch rotating part, a yaw rotating part. Said joint support connected said horizontal motion module of said rear motion module.

In one embodiment, said universal joints of said 4-DoF parallel mechanism are connected together by a linear bearing and a linear shaft that is parallel to the base of said series-parallel hybrid mechanism.

In one embodiment, said 4-DoF parallel mechanism and said series-parallel hybrid mechanism can guarantee two RCM constraints simultaneously.

In one embodiment, said RCM constraints, further including RCM-probe and RCM-needle. Said RCM-probe is located at the patient's anus and said RCM-needle is located on the skin of the patient's perineum.

In one embodiment, said 2-DoF parallel mechanism, its working environment is not limited by the fixed mode of said probe, which includes hand-held, passive mechanism and automatic robotic system.

This invention also provides a method for surgical workflow in transperineal prostate biopsy using MRI and ultrasound images, transrectal ultrasound probe, 3D fusion and registration of the MRI and ultrasound images. In one embodiment, said method comprises: a) acquiring MRI images of the patient's prostate; b) holding said ultrasound probe by said invented device to conduct the specific configuration with said RCM-probe constraint; c) acquiring ultrasound images of the patient's prostate and obtaining the fusion and registration of the MRI and ultrasound images; d) selecting a target area of interest based upon the result of fusion; and directing the said invented device to perform the aiming work with said RCM-needle constraint and RCM-probe constraint; e) inserting said biopsy needle manually through said needle-guide to the target area by surgeons.

In one embodiment, said RCM-probe constraint, further comprising the kinematic modeling to determine the position of RCM-probe relative to the base of said invented device.

In one embodiment, said method further comprises: the method of kinematic modeling of said whole device to realize said RCM-probe constraint and said RCM-needle constraint simultaneously based on said invented device.

In one embodiment, said the RCM-needle position is automatically adjusted by the device upon selection of the target area.

This invention also provides a device for transperineal prostate biopsy in a subject. In one embodiment, said device comprises: a) a first parallel mechanism for generating translational or rotational motion of an ultrasound probe, wherein said translational or rotational motion comprises horizontal, vertical, pitch or yaw motion with RCM-probe constraint; said first parallel mechanism comprises one or more universal joints-motion modules comprising one or more passive joints; b) a series-parallel hybrid mechanism as an end-effector of said first parallel mechanism for housing said ultrasound probe and a biopsy needle-guide; wherein said first parallel mechanism and said series-parallel hybrid mechanism controls configuration of a biopsy needle relative to said subject and performs alignment work with RCM-needle constraint; c) a drive module of said series-parallel hybrid mechanism for generating rotational motion and translational motion of said ultrasound probe along said ultrasound probe's axis; d) a second parallel mechanism of said series-parallel hybrid mechanism for controlling the configuration of said biopsy needle-guide relative to said ultrasound probe and confining trajectory of said biopsy needle to an axial imaging plane of said ultrasound probe; and e) a controller for executing a kinematic modelling method for: i) guaranteeing said RCM-probe constraint based on said first parallel mechanism and said drive module; and ii) guaranteeing said RCM-probe constraint and said RCM-needle constraint simultaneously based on said first parallel mechanism and said series-parallel hybrid mechanism.

In one embodiment, said one or more universal joints-motion modules further comprises two designed linear motion modules to provide 4 DoF; said second parallel mechanism is a 2 DoF mechanism comprising two lifting modules and said biopsy needle-guide, said second parallel mechanism connects to said drive module; wherein said transperineal prostate biopsy device comprises an upper part and a lower part, said series-parallel hybrid mechanism forms said upper part for use as an end-effector of said first parallel mechanism which forms said lower part.

In one embodiment, said two designed linear motion modules comprises a 2-DoF front motion module or a 2-DoF rear motion module.

In one embodiment, said 2-DoF front motion module comprises a horizontal motion module and a vertical motion module, said horizontal motion module connected said vertical motion module, said one or more passive joints connected said horizontal motion module.

In one embodiment, said 2-DoF rear motion module comprises a horizontal motion module and a vertical motion module, said horizontal motion module connected said vertical motion module, said one or more passive joints connected said horizontal motion module.

In one embodiment, said drive module comprises a chamber to fix said ultrasound probe.

In one embodiment, said second parallel mechanism controls the configuration of said biopsy needle-guide relative to said ultrasound probe, said biopsy needle-guide can perform translation and tilt motion relative to said ultrasound probe.

In one embodiment, said drive module comprises a chamber to fix said ultrasound probe and said second parallel mechanism connected said chamber.

In one embodiment, said second parallel mechanism, further comprises an additional mechanism to make said needle perform tilt motion and translation in said axial imaging plane of said ultrasound probe; wherein said drive module comprises a chamber to fix said ultrasound probe and said additional mechanism comprises: a parallelogram mechanism connected to said chamber, said parallelogram mechanism comprises two joints; a needle guide for guiding the biopsy needle which is connected to said parallelogram mechanism; two motors connected to the two joints of said parallelogram mechanism.

In one embodiment, said second parallel mechanism with said biopsy needle-guide moves said biopsy needle in a plane that coincides with said axial imaging plane of said ultrasound probe.

In one embodiment, said one or more passive joints comprises a front universal joint and a rear universal joint.

In one embodiment, said one or more passive joints comprises a front universal joint and a rear universal joint, said front universal joint comprises a joint support, a pitch rotating part, and a yaw rotating part; wherein said joint support is connected to said horizontal motion module of said front motion module.

In one embodiment, said one or more passive joints comprises a front universal joint and a rear universal joint, said rear universal joint, comprises a joint support, a pitch rotating part and a yaw rotating part; wherein said joint support connected said horizontal motion module of said rear motion module.

In one embodiment, said series-parallel hybrid mechanism comprises a base and said one or more passive joints are connected together by a linear bearing and a linear shaft that is parallel to said base.

In one embodiment, said first parallel mechanism and said series-parallel hybrid mechanism can guarantee two RCM constraints simultaneously.

In one embodiment, said RCM constraints comprises a RCM-probe and a RCM-needle, wherein said RCM-probe is to be located at said subject's anus and said RCM-needle is to be located on the skin of said subject's perineum.

In one embodiment, said ultrasound probe is a probe selected from the group consisting of hand-held, passive mechanism and automatic robotic system.

This invention provides a method for transperineal prostate biopsy using the device of this invention. In one embodiment, said method comprises the steps of: a) acquiring MRI images of said subject's prostate; b) holding said ultrasound probe by said transperineal prostate biopsy device to conduct a specific configuration with said RCM-probe constraint; c) acquiring ultrasound images of said subject's prostate; d) fusing and registering the MRI images and ultrasound images to form a fused result; e) selecting a target area of interest based upon said fused result; and directing said device to perform aiming with said RCM-needle constraint and RCM-probe constraint to a RCM-needle position; and f) inserting said biopsy needle manually through said biopsy needle-guide to the target area of interest.

In one embodiment, said device comprises a base and said RCM-probe constraint, further comprising kinematic modelling to determine the position of RCM-probe relative to said base.

In one embodiment, said method further comprises: the method of kinematic modelling of said device to realize said RCM-probe constraint and said RCM-needle constraint simultaneously based on said device.

In one embodiment, said RCM-needle position is automatically adjusted by the device upon selection of the target area of interest.

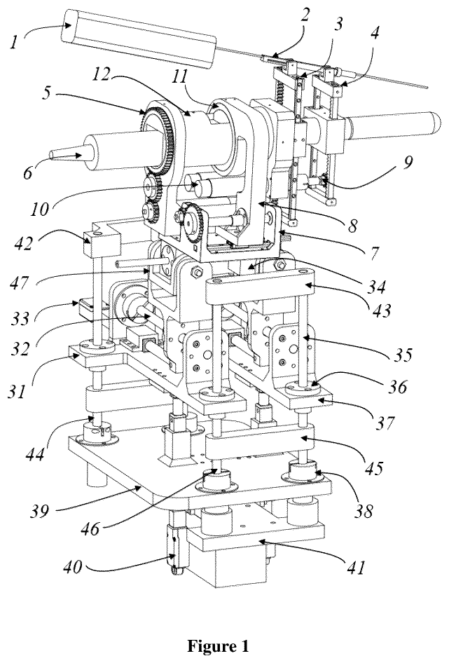

Referring to FIG. 1, a preferred embodiment of the prostate biopsy device is illustrated according to the invention. There are two subassemblies shown in the device: the upper part of the prostate biopsy device and the lower part of the prostate biopsy device. The upper and lower part of the device is connected by the connector 7. The main components of the upper part are the driving mechanism that makes the probe rotate and translate around its own axis, and the parallel mechanism 2-4 that controls the motion of the biopsy needle 1 relative to the probe 6. The lower part of the device, as a designed parallel mechanism, mainly includes two designed universal joints driven by four linear actuators marked 33, 40, 62, and 63 respectively. The whole device can be installed on the other mechanism by its base connector 41.

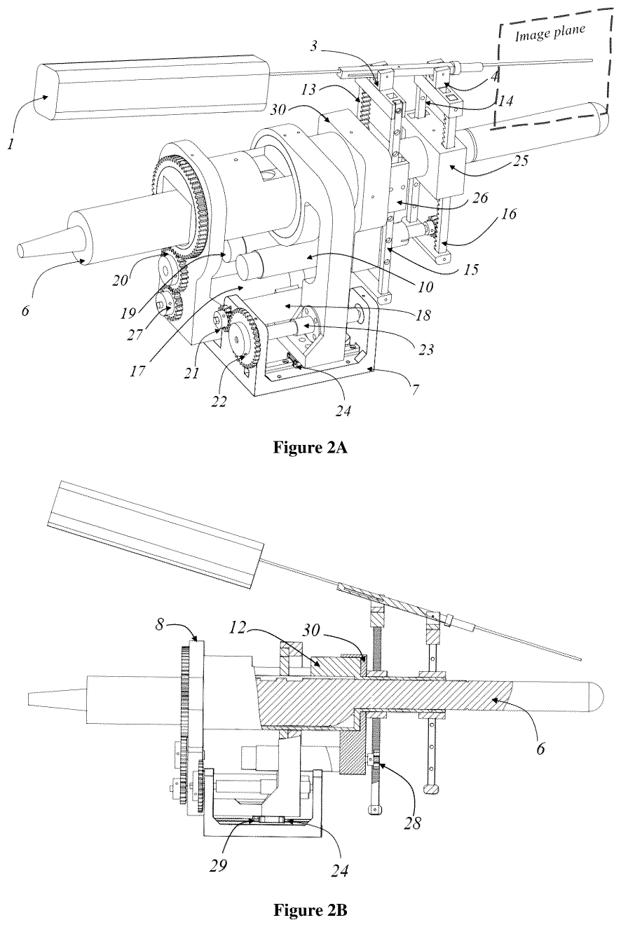

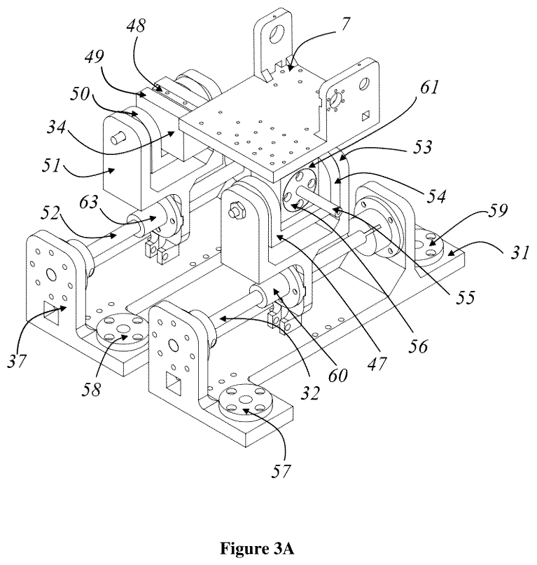

As illustrated in FIG. 2A, the upper part of the prostate biopsy device is shown in detail. The upper part is connected to the lower part of the device by the connector 7 which can be regarded as the base of the upper part. Probe's support 8 is connected to 7 through the linear slide 24. The support 8 can move in translation relative to 7, which is driven by the motor 18 through the gears 21 and 22 and the lead screw 23. The screw nut is fixed on the support 8. The probe 6 is capable of rotational movement relative to the part 8 along its axis, driven by the motor 17 and a set of three gears 27, 20 and 5 that reduce the speed and increase the torque produced by the motor 17. The probe 6 is fixed by the chamber 12 which is connected with the support 8 by the bearing 11. The parallel mechanism consists of 2-4 that control the motion of the biopsy needle 1 relative to the probe 6. This motion includes translation and tilt relative to the probe, which are driven by motors 10 and 19. The racks 13 and 16 and the linear guides 15 and 14 convert the rotary motion of the motors into translational motion, moreover, two sets of racks and linear guides are fixed on the chamber 12 through parts 26 and 25, while two sets of motors are fixed on the chamber 12 through parts 30. The needle guide 2 is connected with the lifting modules 3 and 4 of the parallel mechanism. Through the relative movement of the motor 10 and motor 19, the needle-guide's movement of tilt and translation in the image plane is realized.

FIG. 2B is a partial sectional view of the upper part of the device, which can better reflect the connection relationship mentioned above between the support 8, the chamber 12, the part 30 and the probe 6. The parallel mechanism of the upper part of the device is driven by two motors, the rotation motion is transformed to translation motion by two gears 9, 28 and racks. Through the linear guides 29 and 24, the support 8 is advanced and retracted along the axis of the probe in a linear way.

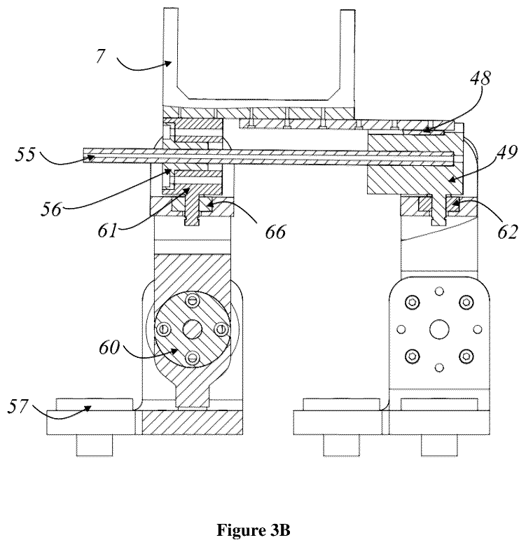

FIG. 3A illustrates an orthogonal view of the passive motion module of the lower part of the prostate biopsy device. This parallel mechanism mainly includes two designed universal joints 47 and 34. The universal joint 47 is mainly composed of a support 53, a pitch rotating part 54 and a yaw rotating part 61, and two linear bearings 60 and 53. The linear bearing 60 is fixed under the support 53, and the linear bearing 56 is fixed inside the yaw rotating part 61. Among them, the pitch rotating part 54 can perform pitch rotation relative to the supporting part 53, and the yaw rotating part 61 can perform yaw rotation relative to the pitch rotating part 54. In addition, the configuration of universal joint 34 is slightly different from that of universal joint 47. Similarly, the main components of the universal joint 34 are the support 51, the pitch rotating part 50 and the yaw rotating part 49, and a linear bearing 64. The linear bearing 64 is fixed under the support 51. In addition, the pitch rotating part 50 can perform pitch rotation relative to the support 51, and the yaw rotating part 49 can perform yaw rotation relative to the part pitch rotating 50. Unlike the U-joint (universal joint) 47, the yaw rotating part 49 of the U-joint 34 has a slider of a linear guide 48 mounted inside.

One end of the linear shaft 55 passes through the interior of the part 49 and is fixed, and the other end passes through the linear bearing 56 inside the part 53 of the U-joint 47. The linear shaft 55 can slide along the axis of the linear bearing 56. The base 7 of the upper part of the device is fixed on the part 61. Moreover, one end of the linear guide 48 is fixed under the base 7, and the other end can slide along the sliding slider inside the part 49. Therefore, the linear guide 48 and the linear shaft 55 are always parallel.

The two linear shafts 52 and 32 placed horizontally pass through two linear bearings 64 and 60 that are fixed under the U-joint 47 and 34, respectively. In addition, these two linear shafts 52 and 32 are fixed to the connecting plates 31 and 37, respectively. Therefore, the two U-joints 47 and 34 can do telescopic motion along the direction of the linear shafts in a linear way that is driven by the linear actuators 33 and 63 respectively. The ends of the linear actuators 33 and 63 are connected with the support 51 and the support 53, and the bases of the linear actuators are fixed on the connection plates 31 and 37. When the telescopic amount of the two linear actuators 33 and 63 is equal, the two U-joints 47 and 34 simultaneously perform horizontal translation; when the telescopic amounts of the two linear actuators 33 and 63 are not equal, the relative positions of the two U-joints 47 and 34 in the horizontal direction change, which causes the yaw rotors 49 and 61 to rotate, therefore the base 7 also exhibits the yaw movement.

The two linear bearings 57 and 59 placed vertically are fixed to both ends of the connecting plate 31, and similarly, linear bearings 58 and 36 are fixed to both ends of the connecting plate 37. The linear shafts 44 and 46 pass through the linear bearings 57 and 59 respectively, and these two linear shafts are also fixed on the base 39. Likewise, the linear shafts 65 and 35 pass through the linear bearings 58 and 36, respectively, and are also fixed to the base 39. Therefore, two U-joints 47 and 34 and two connecting plates 31 and 37 constitute two modules that moves in the vertical direction, which are driven by the linear actuators 40 and 62. When the telescopic amounts of the two linear actuators 40 and 62 are equal, the two U-joints 47 and 34 simultaneously perform translational movement in the vertical direction; when the telescopic amounts of the two linear actuators 40 and 62 are not equal, the relative positions of the two U-joints 47 and 34 in the vertical direction will change, causing the pitch rotating parts 47 and 50 to rotate. Therefore, the base 7 also shows the pitch movement. Therefore, through this designed parallel structure, the upper part of the device can be made to achieve vertical and horizontal translational motions, as well as pitch and yaw rotations.

FIG. 3B is a partial sectional view of the passive motion module of the lower part of the prostate biopsy device, which can better reflect the connection relationship mentioned above between the components (the yaw rotating parts 61 and 49, the pitch rotating parts 54 and 50, the linear shaft 55, the linear guide 48 and the base 7) of the two universal joints 47 and 34. Since the yaw rotating part 61 and 49 are to be rotated relative to the parts 54 and 50, it is necessary to connect with each other through the bearing 66 and 62, respectively. The linear guide 48 increases the stiffness and stability of the base 7.

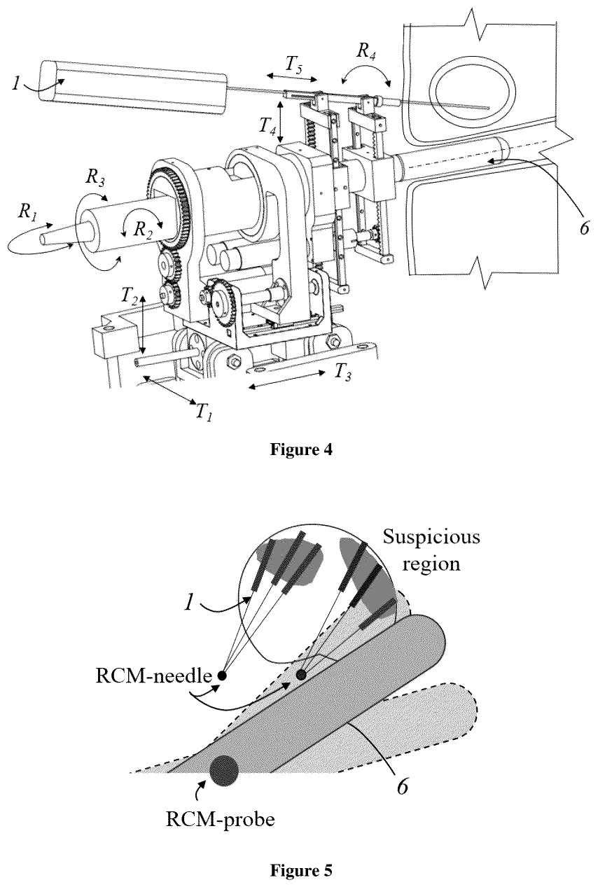

The kinematic diagram of the prostate biopsy device with partial view according to the present invention is presented in FIG. 4. At its zero configuration, the axial direction of the probe 6 is horizontal and perpendicular to the telescopic direction of the linear actuators 33 and 63. This device has a total of 8 degrees of freedom. Moreover, the needle 1 has translational motion (T5) relative to the needle-guide 2, which is driven by surgeons when the prostate biopsy device has completed the aiming work. The translation (T1 and T2) and the yaw (R1) as well as the pitch (R2) are controlled by the four linear actuators. The probe 6 can perform rotational (R3) and translational motions (T3) relative to its own axis, that are driven by motor 17 and motor 18 with a set of gears. In summary, the probe fixed on the device will have 6 degrees of freedom, including three rotational movements and translational motions in three directions. In addition, the needle guide 2 is always located on the axial ultrasonic imaging plane of the probe 6, which can ensure that the needle can be displayed to the doctor on the ultrasonic image in real time. Furthermore, the position of the needle guide 2 related to the probe 6 could be determined by a two degree of freedom parallel mechanism. Through the relative motion of motor 10 and motor 19, the translation (T4) and tilt motion (R4) of needle-guide relative to probe can be realized. When the system is working, the front end of the probe 6 is inserted into the rectum of the patient, and the back end is fixed on the device. After the doctor selects the target point and then the system completes the positioning work, the doctor performs the final insertion operation (T5). The biopsy needle is inserted into the prostate through the patient's perineum to complete the biopsy sampling. Therefore, in this mode, the device assists the doctor to perform transperineal prostate biopsy to the greatest extent while ensuring safety.

FIG. 5 shows the RCM constraints of the device during the biopsy process. Based on the flexible structure, the whole system includes two RCM (remote center of motion) constraints, namely RCM-needle and RCM-probe. The RCM-needle position could be movable relative to the probe 6 under different working conditions. The RCM-probe is at the patient's anus, and there is no relative displacement between the probe and anus, which can reduce the patient's discomfort. The RCM-needle is located on the skin of the patient's perineum. It is worth mentioning that the RCM-needle is in the axial imaging plane of the probe 6. As shown in the figure, the RCM-needle is usually located on both sides to avoid important nerves in the center. A biopsy usually requires more than 20 needle sticks. Therefore, RCM-needle restraint can reduce superficial trauma to the patient's skin, thereby reducing the risk of infection. In addition, in this way, the use of anesthetics can be reduced and the area of anesthesia of the patient can be reduced as well.

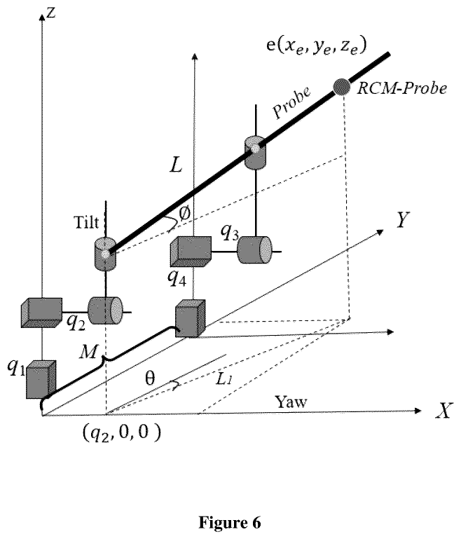

FIG. 6 illustrates a kinematic diagram of the lower part of the device. Since the RCM-probe is expected to be at the patient's anus, the forward and inverse kinematics model of the parallel mechanism can be established based on the geometric constraints. It is worth mentioning that the geometric constraints include the distance denoted as M between the linear actuator 40 and linear actuator 62, and the initial distance between the position of the RCM-probe and linear actuator 62 denoted as L. The position of the anus (denoted as e(xₑ, yₑ, zₑ)) relative to the base of the device is determined according to the actual working condition. Given the position of the anus, the pitch and yaw angles (denoted as ø and θ) can be determined by the expansion values of the linear actuators 40 and 33 (denoted as q₁ and q₂), thus (θ, ø)=f (q₁, q₂). And then based on the geometric constraints, the expansion values of the linear actuators 62 and 63 (denoted as q₄ and q₃) can be determined, thus (q₁, q₂)=f(q₃, q₄).

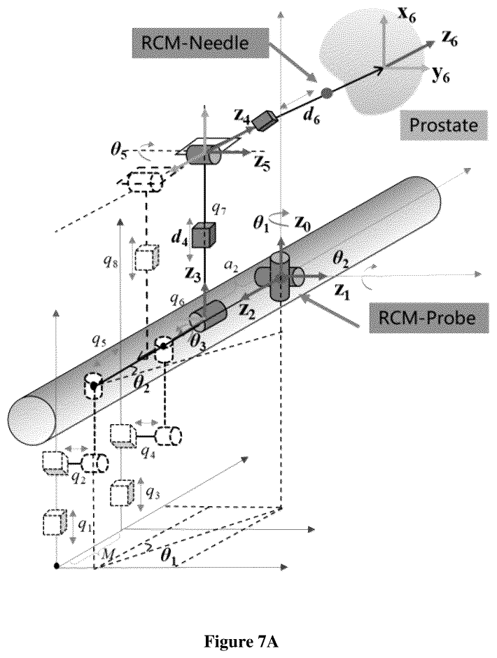

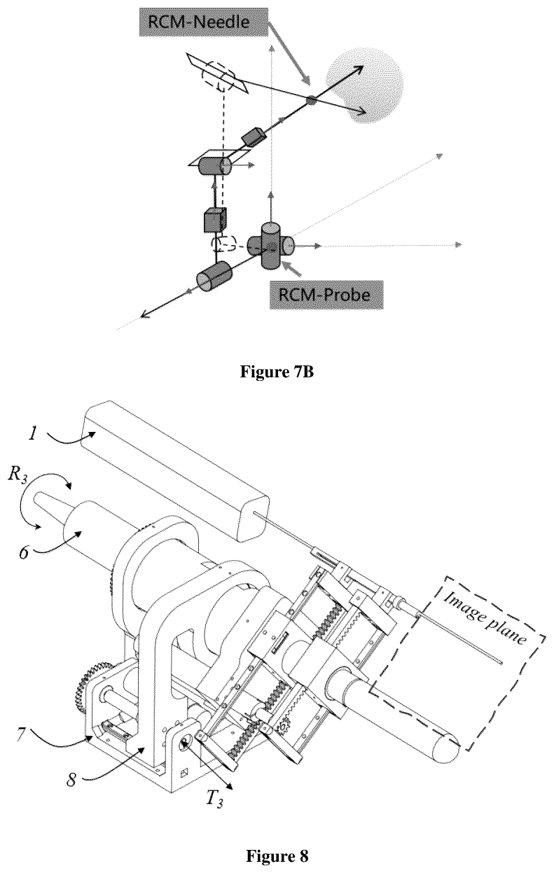

FIG. 7A is the kinematic diagram of the whole device. To deal with the RCM-probe and RCM-needle simultaneously, the kinematics model as shown in the figure is established. When the position of the anus relative to the base of the device is determined, it is assumed that it is the origin of a 6-DOF manipulator, which is represented by the coordinate 0. It is worth noting that there is no relative displacement between the probe and the patient's anus. The translation motion of the upper part of the device is denoted as q₅. Thus, based on the lower parallel mechanism and its kinematic analysis mentioned above, the yaw and pitch rotation denoted as θ₁ and θ₂ can be represented as θ₁=f(q₁, q₂, q₅) and θ₂=f(q₃, q₄, q₅), respectively. The rotation along the probe axis is denoted as q₅, which can provide the roll motion denoted as θ₃, thus θ₃=f(q₆). The translation and tilt motion of the needle relative to the probe mentioned above are denoted as d₄ and θ₅, which are determined by the relative displacement of the motors of the parallel mechanism of the upper part of the device denoted as q₇ and q₈. Thus, d₄=f(q₇,q₈) and θ₅=f(q₇,q₈). The needle insertion is regarded as a passive joint of the manipulator denoted as d₆. Therefore, the forward kinematic model can be established based on the diagram, which can describe the relationship between the position of the patient's anus and the position of the cancer region targets. And the RCM-needle can be realized by adding constraints to the kinematic equations:

$${p}_{r c m} = {P}_{5}^{0} + λ ( {P}_{6}^{0} - {P}_{5}^{0} ) and \dot{{p}_{r c m}} = {J}_{r c m} ( \begin{matrix}\dot{q} \\ \dot{λ}\end{matrix} ) ,$$

where p_{rcm} represents the position of the RCM-needle.

$${P}_{5}^{0} and {P}_{6}^{0}$$

are the base of the coordinate attached to the front joint of the parallel mechanism and cancer region targets. J_{rcm} denotes the Jacobian matrix and λ is a parameter that can adjust the position of RCM-needle. The parameter q=[θ₁, θ₂, θ₃, d₄, θ₅, d₆].

FIG. 7B are motion diagram of the whole device under two RCM constraints at two different configurations during the process of targeting different regions. As illustrated the diagram, the whole device can guarantee RCM-probe and RCM-needle simultaneously.

FIG. 8 gives an orthogonal view of the upper part of the prostate biopsy device in a configuration when the probe 6 performs rotation (R3) and translation (T3) relative to its axis. It can be seen from the perspective view of this configuration that when the probe rotates, the needle also rotates through mechanical constraints, and the needle 1 is always on the imaging plane of the probe axis. This characteristic ensures safety, that is, when the needle is inserted into the patient body, it will always be observed.

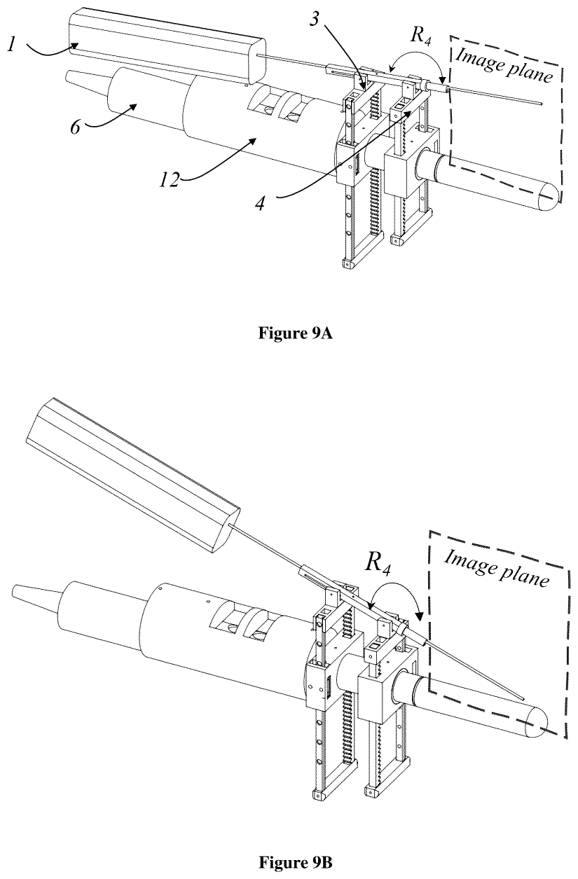

FIGS. 9A and 9B are the orthogonal views of the needle motion module of the upper part of the prostate biopsy device in two different configurations when the needle 1 performs the tilt motion (R4) relative to the probe 6 of the device. The needle-guide 2 and the modules 3 and 4 of the parallel structure, and the chamber 12 form a closed kinematic chain. The rotation angle of the needle-guide 2 is determined by the relative positions of modules 3 and 4 of the parallel structure, and is always in the imaging plane of the probe axis. The rotation range is ±20 degrees. With this design, the drive motors 9 and 19 of modules 3 and 4 can be placed farther away from the needle-guide.

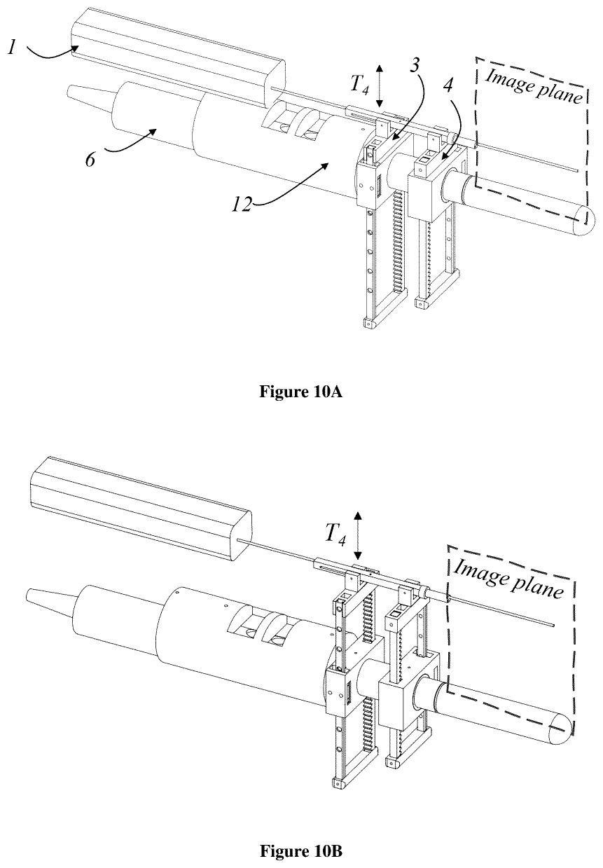

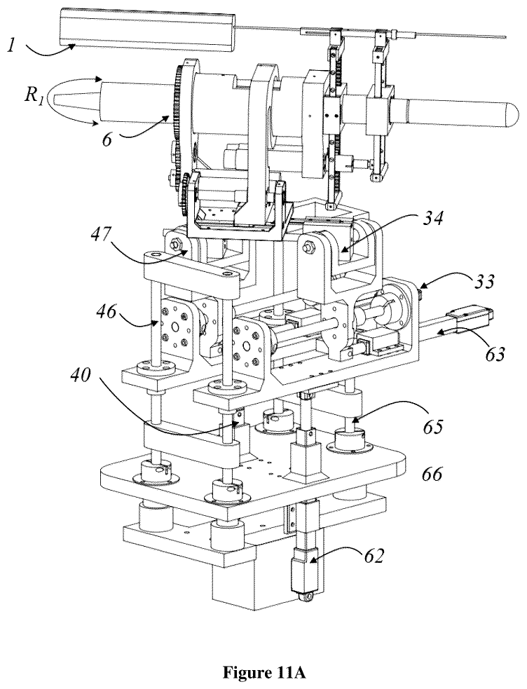

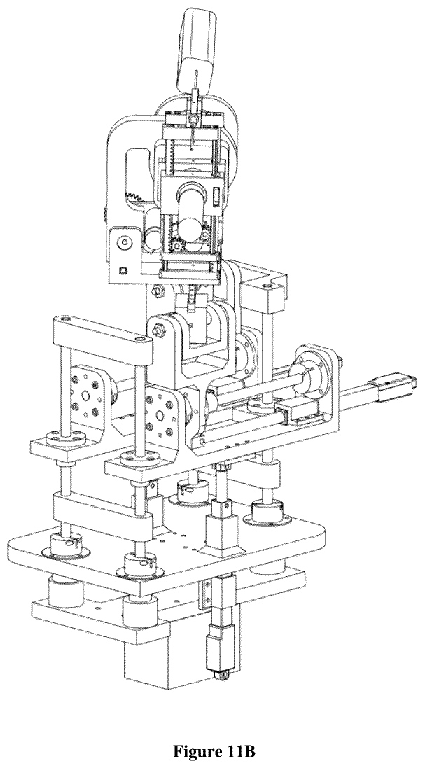

FIGS. 10A and 10B are the orthogonal views of the needle motion module of the upper part of the prostate biopsy device in two different configurations when the needle 1 performs the translation motion (T4) relative to the probe 6 of the device. The translation range of the needle-guide 2 is determined by the relative positions of modules 3 and 4 of the parallel structure. When the telescopic distances of modules 3 and 4 are equal, the needle-guide 2 can rise or fall relative to the probe 6.

FIGS. 11A and 11B are the orthogonal views of the prostate biopsy device in two different configurations when it performs the yaw motion (R1). The upper part of the device can be regarded as the end-effector of the lower parallel part of the device. Furthermore, the base of the upper part of the device is regarded as the end-effector of the lower parallel mechanism. Based on the function of the device and the actual working conditions mentioned above, the RCM-probe is expected to be at the patient's anus. And there is no relative displacement between the probe and the patient's anus. Therefore, when the device performs the yaw rotation, the relationship of the relative expansion displacement of the linear actuators 62 and 40 can be determined.

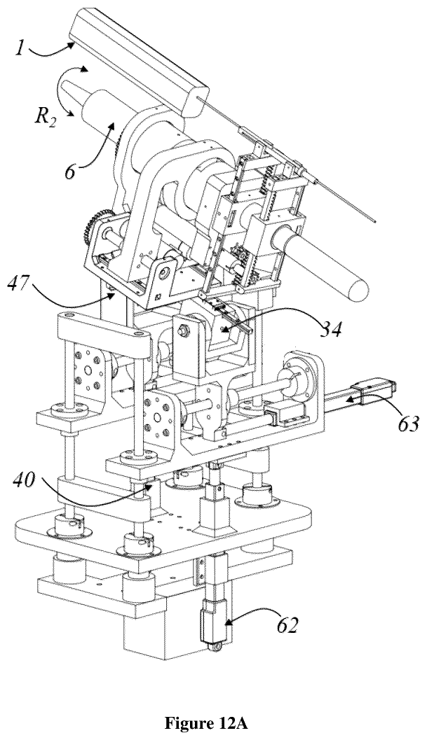

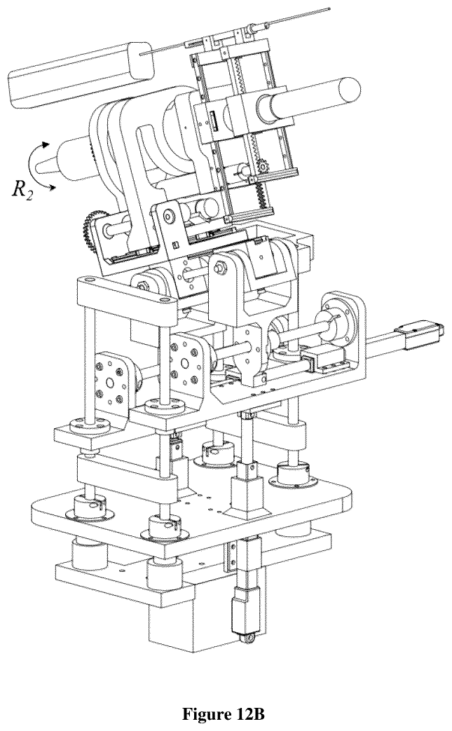

FIGS. 12A and 12B are the orthogonal views of the prostate biopsy device in two different configurations when it performs the pitch motion (R2). The yaw motion and the pitch motion are decoupled, and the device can also do yaw rotation while doing pitch rotation. As illustrated in FIG. 9, the RCM-probe is expected to be at the patient's anus. Therefore, the relationship of the relative expansion displacement of the linear actuators 63 and 33 can be determined to meet the requirement of the RCM-probe.

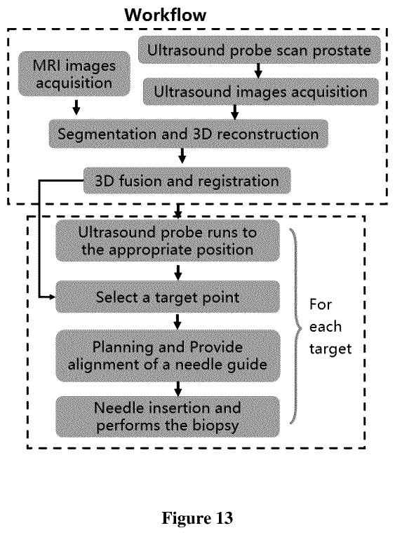

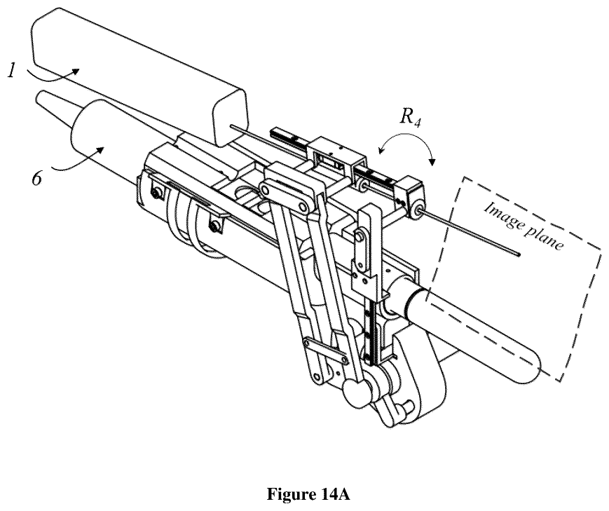

FIG. 13 shows the workflow with the invented device for transperineal prostate biopsy. First, the ultrasonic probe is held by the device to conduct the specific configuration with RCM-probe constraint and then a series of ultrasound images are acquired. It should be noted that the MRI images are always acquired before the process of the biopsy. Next, based on the fusion and registration of the MRI and ultrasound images, the cancer suspicious regions are targeted. Consequently, the device performs the aiming work with two RCM constraints, at the same time, the plane of movement of the needle will always be limited to the imaging plane of the probe. Finally, for each target, the surgeon performs the manual needle insertion through the perineal to the prostate of the patient.

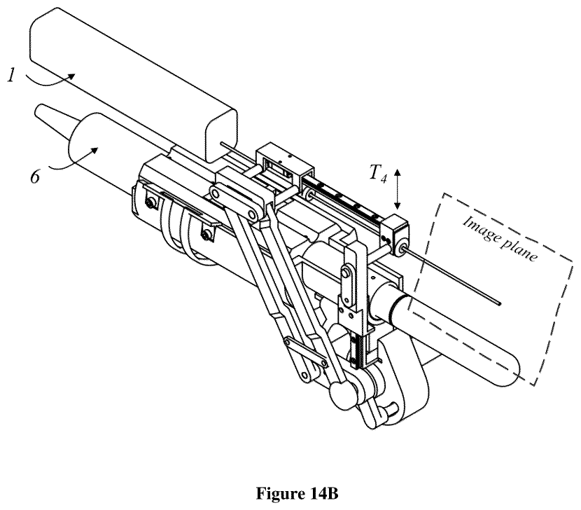

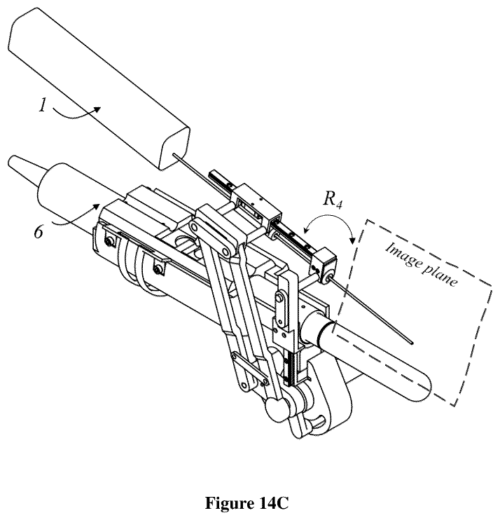

FIGS. 14A to 14C show another mechanism of the needle motion module that can make the needle perform the tilt motion and translation in the image plane relative to the probe of the device at different configurations. Since the degrees of freedom of the parallel mechanism are two, it is driven by two actuators same as the parallel mechanism mentioned above. A parallelogram mechanism is connected to the chamber that fixes the probe, and the needle-guide for guiding the biopsy needle is connected to the parallelogram mechanism. In addition, the motors are connected to the two joints of the parallelogram mechanism.

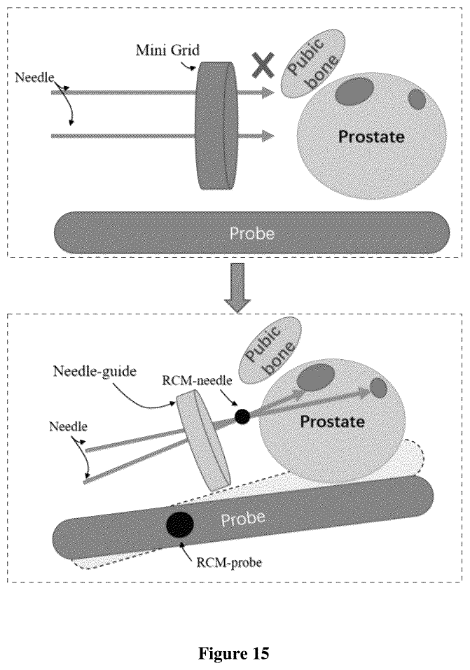

FIG. 15 shows the advantageous functions of invented device compared to traditional methods during transperineal prostate biopsy procedure. Because of the existence of bone, in some methods, the probe can only rotate around its own axis, so when the cancer area is located in the upper area of the prostate, the biopsy needle cannot successfully reach the cancer area through the mini grid parallel to the probe; In addition, for some devices, such as monalisa, the needle has multiple degrees of freedom, but the needle cannot be limited to the image plane of the probe. The device can comprehensively solve the above problems with its flexible structure and kinematic modeling method mentioned above.

While the detail of this invention has been described above, it should be noted that the invention is not limited thereto but can be practiced in various ways as illustrated in the following claims.

What is claimed is:

1. A device for transperineal prostate biopsy in a subject, comprising:
a. a first parallel mechanism for generating translational or rotational motion of an ultrasound probe, wherein said translational or rotational motion comprises horizontal, vertical, pitch or yaw motion with RCM-probe constraint; said first parallel mechanism comprises one or more universal joints-motion modules comprising one or more passive joints;
b. a series-parallel hybrid mechanism as an end-effector of said first parallel mechanism for housing said ultrasound probe and a biopsy needle-guide; wherein said first parallel mechanism and said series-parallel hybrid mechanism controls configuration of a biopsy needle relative to said subject and performs alignment work with RCM-needle constraint;
c. a drive module of said series-parallel hybrid mechanism for generating rotational motion and translational motion of said ultrasound probe along said ultrasound probe's axis;
d. a second parallel mechanism of said series-parallel hybrid mechanism for controlling the configuration of said biopsy needle-guide relative to said ultrasound probe and confining trajectory of said biopsy needle to an axial imaging plane of said ultrasound probe; and
e. a controller for executing a kinematic modelling method for:
i) guaranteeing said RCM-probe constraint based on said first parallel mechanism and said drive module; and
ii) guaranteeing said RCM-probe constraint and said RCM-needle constraint simultaneously based on said first parallel mechanism and said series-parallel hybrid mechanism.

2. The device of claim 1, wherein
said one or more universal joints-motion modules further comprises two designed linear motion modules to provide 4 DoF;
said second parallel mechanism is a 2 DoF mechanism comprising two lifting modules and said biopsy needle-guide, said second parallel mechanism connects to said drive module;
wherein said transperineal prostate biopsy device comprises an upper part and a lower part, said series-parallel hybrid mechanism forms said upper part for use as an end-effector of said first parallel mechanism which forms said lower part.

3. The device of claim 2, wherein said two designed linear motion modules comprises a 2-DoF front motion module or a 2-DoF rear motion module.

4. The device of claim 3, wherein said 2-DoF front motion module comprises a horizontal motion module and a vertical motion module, said horizontal motion module connected said vertical motion module, said one or more passive joints connected said horizontal motion module.

5. The device of claim 3, wherein said 2-DoF rear motion module comprises a horizontal motion module and a vertical motion module, said horizontal motion module connected said vertical motion module, said one or more passive joints connected said horizontal motion module.

6. The device of claim 1, wherein said drive module comprises a chamber to fix said ultrasound probe.

7. The device of claim 2, wherein said second parallel mechanism controls the configuration of said biopsy needle-guide relative to said ultrasound probe, said biopsy needle-guide can perform translation and tilt motion relative to said ultrasound probe.

8. The device of claim 7, wherein said drive module comprises a chamber to fix said ultrasound probe and said second parallel mechanism connected said chamber.

9. The device of claim 7, wherein said second parallel mechanism, further comprises an additional mechanism to make said needle perform tilt motion and translation in said axial imaging plane of said ultrasound probe; wherein said drive module comprises a chamber to fix said ultrasound probe and said additional mechanism comprises:
a parallelogram mechanism connected to said chamber, said parallelogram mechanism comprises two joints;
a needle guide for guiding the biopsy needle which is connected to said parallelogram mechanism; and
two motors connected to the two joints of said parallelogram mechanism.

10. The device of claim 2, wherein said second parallel mechanism with said biopsy needle-guide moves said biopsy needle in a plane that coincides with said axial imaging plane of said ultrasound probe.

11. The device of claim 1, wherein said one or more passive joints comprises a front universal joint and a rear universal joint.

12. The device of claim 4, wherein said one or more passive joints comprises a front universal joint and a rear universal joint, said front universal joint comprises a joint support, a pitch rotating part, and a yaw rotating part; wherein said joint support is connected to said horizontal motion module of said front motion module.

13. The device of claim 4, wherein said one or more passive joints comprises a front universal joint and a rear universal joint, said rear universal joint, comprises a joint support, a pitch rotating part and a yaw rotating part; wherein said joint support connected said horizontal motion module of said rear motion module.

14. The device of claim 2, wherein said series-parallel hybrid mechanism comprises a base and said one or more passive joints are connected together by a linear bearing and a linear shaft that is parallel to said base.

15. The device of claim 2, wherein said first parallel mechanism and said series-parallel hybrid mechanism can guarantee two RCM constraints simultaneously.

16. The device of claim 14, wherein said RCM constraints comprises a RCM-probe and a RCM-needle, wherein said RCM-probe is to be located at said subject's anus and said RCM-needle is to be located on the skin of said subject's perineum.

17. The device of claim 9, wherein said ultrasound probe is a probe selected from the group consisting of hand-held, passive mechanism and automatic robotic system.

18. A method for transperineal prostate biopsy using the device of claim 1, comprising the steps of:
a. Acquiring MRI images of said subject's prostate;
b. Holding said ultrasound probe by said transperineal prostate biopsy device to conduct a specific configuration with said RCM-probe constraint;
c. Acquiring ultrasound images of said subject's prostate;
d. fusing and registering the MRI images and ultrasound images to form a fused result;
e. Selecting a target area of interest based upon said fused result; and directing said device to perform aiming with said RCM-needle constraint and RCM-probe constraint to a RCM-needle position; and
f. Inserting said biopsy needle manually through said biopsy needle-guide to the target area of interest.

19. The method of claim 18, wherein said device comprises a base and said RCM-probe constraint, further comprising kinematic modelling to determine the position of RCM-probe relative to said base.

20. The method of claim 18, further comprising: the method of kinematic modelling of said device to realize said RCM-probe constraint and said RCM-needle constraint simultaneously based on said device.

21. The method of claim 18, wherein said RCM-needle position is automatically adjusted by the device upon selection of the target area of interest.
